(19) United States
(12) Patent Application Publication
(10) Pub. No.: US 2007/0119928 A1
(43) Pub. Date: May 31, 2007

(54) Generating a nutraceutical request from an inventory

(76) Inventors: Edward K.Y. Jung, Bellevue, WA (US); Royce A. Levien, Lexington, MA (US); Robert W. Lord, Seattle, WA (US); Mark A. Malamud, Seattle, WA (US); John D. Rinaldo JR., Bellevue, WA (US); Clarence T. Tegreene, Bellevue, WA (US); Lowell L. Wood JR., Livermore, CA (US)

Correspondence Address: SEARETE LLC;CLARENCE T. TEGREENE, 1756 - 114TH AVE., S.E., SUITE 110, BELLEVUE, WA, 98004, US

(21) Appl. No.: 11/291,482

(22) Filed: Nov. 30, 2005

Related U.S. Application Data
(63) Continuation-in-part of application No. 11/283,548, filed on Nov. 17, 2005.

(51) Int. Cl. G06Q 30/00

(52) U.S. Cl. 235/385

(57) ABSTRACT

A method, system, and computer program product are described for registering a dispensation from an inventory and responding to the registering by indicating a nutraceutical request quantity partly based on the inventory and partly based on a consumption regimen.

### RELATED APPLICATIONS

1. For purposes of the USPTO extra-statutory requirements, the present application constitutes a continuation-in-part of U.S. patent application Ser. No. 11/283,548, entitled PROVIDING ASSISTANCE RELATED TO HEALTH, naming Edward K. Y. Jung, Joyce A. Levien, Robert W. Lord, Mark A. Malamud, John D. Rinaldo, Jr., Clarence T. Tegreene and Lowell L. Wood, Jr. as inventors, filed Nov. 17, 2005, which is currently co-pending, or is an application of which a currently co-pending application is entitled to the benefit of the filing date.

2. For purposes of the USPTO extra-statutory requirements, the present application constitutes a continuation-in-part of U.S. patent application Ser. No. ______, entitled TESTING-DEPENDENT ADMINISTRATION OF A NUTRACEUTICAL, naming Edward K. Y. Jung, Royce A. Levien, Robert W. Lord, Mark A. Malamud, John D. Rinaldo, Jr., Clarence T. Tegreene an dLowell L. Wood, Jr. as inventors, filed contemporaneously herewith, which is currently co-pending, or is an application of which a currently co-pending application is entitled to the benefit of the filing date.

### CROSS-REFERENCE TO RELATED APPLICATIONS

The present application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed application(s) (the “Related Applications”) to the extent such subject matter is not inconsistent herewith; the present application also claims the earliest available effective filing date(s) from, and also incorporates by reference in its entirety all subject matter of any and all parent, grandparent, great-grandparent, etc. applications of the Related Application(s) to the extent such subject matter is not inconsistent herewith. The United States Patent Office (USPTO) has published a notice to the effect that the USPTO's computer programs require that patent applicants reference both a serial number and indicate whether an application is a continuation or continuation in part. The present applicant entity has provided below a specific reference to the application(s)from which priority is being claimed as recited by statute. Applicant entity understands that the statute is unambiguous in its specific reference language and does not require either a serial number or any characterization such as “continuation” or “continuation-in-part.” Notwithstanding the foregoing, applicant entity understands that the USPTO's computer programs have certain data entry requirements, and hence applicant entity is designating the present application as a continuation in part of its parent applications, but expressly points out that such designations are not to be construed in any way as any type of commentary and/or admission as to whether or not the present application contains any new matter in addition to the matter of its parent application(s).

### SUMMARY

An embodiment provides a method. In one implementation, the method includes but is not limited to registering a dispensation from an inventory and responding to the registering by indicating a nutraceutical request quantity partly based on the inventory and partly based on a consumption regimen. In addition to the foregoing, other communication method aspects are described in the claims, drawings, and text forming a part of the present disclosure.

In one or more various aspects, related systems include but are not limited to circuitry and/or programming for effecting the herein referenced method aspects; the circuitry and/or programming can be virtually any combination of hardware, software, and/or firmware configured to effect the herein-referenced method aspects depending upon the design choices of the system designer.

An embodiment provides a system. In one implementation, the system includes but is not limited to circuitry for registering a dispensation from an inventory and a module for responding to the registering by indicating a nutraceutical request quantity partly based on the inventory and partly based on a consumption regimen. In addition to the foregoing, other computer program product aspects are described in the claims, drawings, and text forming a part of the present disclosure.

An embodiment provides another system. In one implementation, the other system includes but is not limited to a computing device and one or more instructions that when executed by the computing device cause the computing device to perform at least one of registering a dispensation from an inventory and responding to the registering by indicating a nutraceutical request quantity partly based on the inventory and partly based on a consumption regimen. In addition to the foregoing, other computer program product aspects are described in the claims, drawings, and text forming a part of the present disclosure.

An embodiment provides a computer program product. In one implementation, the computer program product includes but is not limited to a signal-bearing medium bearing at least one of (a) one or more instructions for registering a dispensation from an inventory and (b) one or more instructions for responding to the registering by indicating a nutraceutical request quantity partly based on the inventory and partly based on a consumption regimen.

In addition to the foregoing, various other embodiments are set forth and described in the text (e.g., claims and/or detailed description) and/or drawings of the present description.

The foregoing is a summary and thus contains, by necessity, simplifications, generalizations and omissions of detail; consequently, those skilled in the art will appreciate that the summary is illustrative only and is not intended to be in any way limiting. Other aspects, features, and advantages of the devices and/or processes described herein, as defined by the claims, will become apparent in the detailed description set forth herein.

### BRIEF DESCRIPTION OF THE FIGURES

FIG. 1 shows an operational flow representing example operations that produce an indication of an amount.

FIG. 2 shows an example system in schematic form, a hardware implementation able to perform variants of the flow of FIG. 1.

FIG. 3 shows another example system able to perform the flow of FIG. 1 and many similar variations.

FIG. 4 shows another example system able to perform the flow of FIG. 1 and many similar variations.

FIG. 5 shows various optional features of the flow of FIG. 1.

FIG. 6 shows another example system able to perform many variants of the above-described flows.

FIG. 7 shows various optional features of the flow of FIG. 1 or 5.

FIG. 8 shows various optional features of the flow of FIG. 1, 5, or 7.

FIG. 9 shows further optional features of the system of FIG. 4.

FIG. 10 shows various optional features of the flow of FIG. 1, 5, 7 or 8.

FIG. 11 shows a system that includes a signal-bearing medium that can comprise or interact with a conduit, a disk, an integrated circuit, or a computing device.

FIG. 12 shows other optional features of the above-described flows.

FIG. 13 shows still more optional features of the above-described flows.

### DETAILED DESCRIPTION

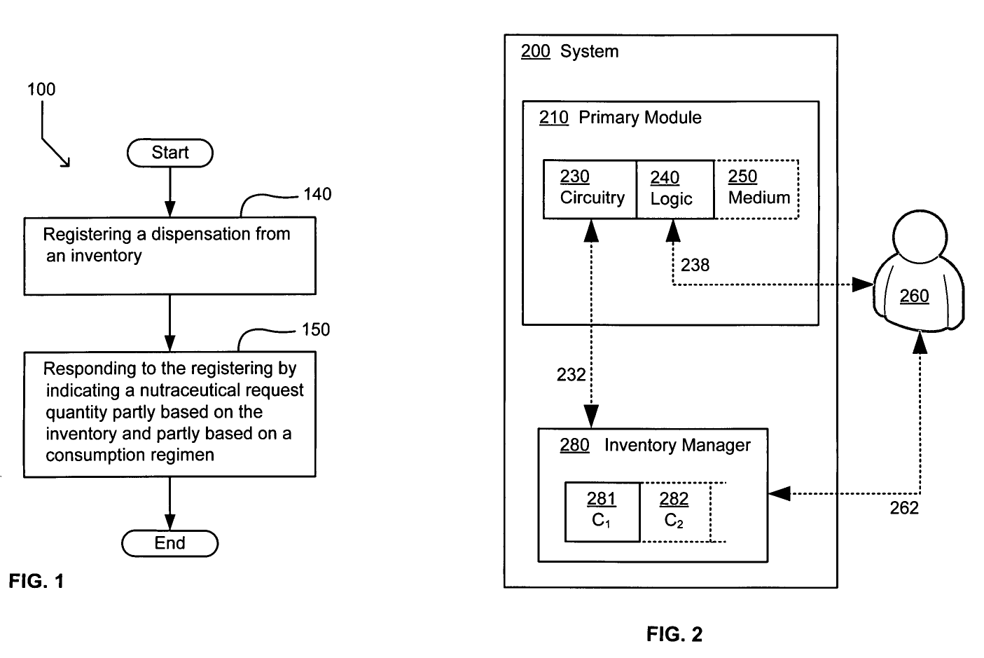

FIG. 1 shows an operational flow 100 representing example operations that produce an indication of an amount of a request quantity, such as by indicating a rate or otherwise expressing a quantity that includes a nutraceutical. Flow 100 and other embodiments as described below systemize regimens that include one or more of these components, facilitating or enabling a user's implementation of a regimen.

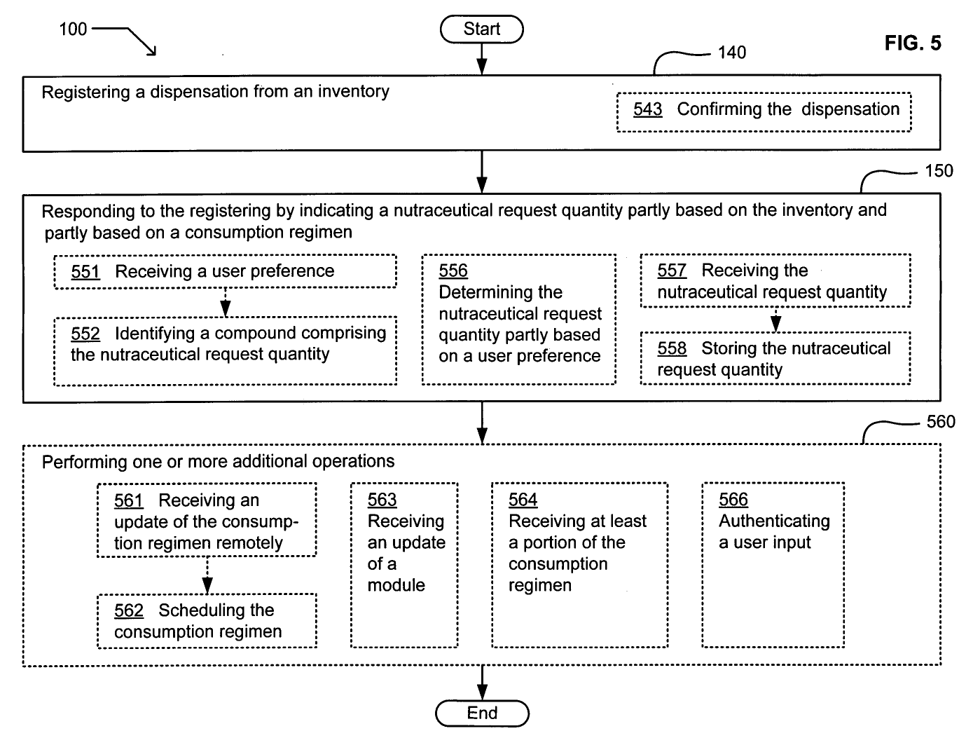

After a start operation, operational flow 100 moves to operation 140, comprising registering a dispensation from an inventory. Flow 100 then moves to operation 150, comprising responding to the registering by indicating a nutraceutical request quantity partly based on the inventory and partly based on a consumption regimen. The indication can be a display or other transmission, for example. The consumption regimen can optionally contain one or more regimens or regimen components, such as seasonal or occasional or other conditional regimen(s). Alternatively or additionally, the consumption regimen can comprise a component of a master regimen that also includes a non-consumption component. FIGS. 5, 7, 8, 10, and 12 below include many variations of operational flow 100. In various embodiments such as these, of course, flow 100 can include additional operations or variations in the sequence of operations.

Referring now to FIG. 2, there is shown an example system 200 in schematic form, a hardware implementation able to perform variants of flow 100 as described below. Primary module 210 includes circuitry 230 for receiving at least an indication of the registration (via linkage 232, e.g.) of a state of a subject (an animal or other organism, e.g.). Primary module 210 further includes logic 240 (such as a processor or programmable logic, e.g.) for indicating the request quantity (via interface 238, e.g.) partly based on the dispensation or other aspect of the inventory, and partly based on the consumption regimen. Primary module 210 can further include medium 250 accessible by at least logic 240, as described below. (The dashed outline of medium 250 signifies that some embodiments are specifically contemplated to exclude this feature, and others are contemplated to include it.)

Linkage 232 is similarly indicated as optional. Even if system 200 is merely a kit or physical structure with no communication linkage 232, for example, it can be advantageous for user 260 to access primary module 210 in proximity to inventory manager 280. Linkage 232 can include one or more of analog data, digital data, or a measurable physical property such as a distance or similar geometry. Alternatively or additionally, linkage 232 can comprise a conduit bearing one or more instructions that can be stored in medium 250, for example, or can be executed by logic 240 to perform one or more variations of flow 100 such as those shown in FIGS. 5, 7, 8, 10, and 12. Alternatively or additionally, information can be conveyed to primary module on a ticket or a similar printed record or memory device that can be deposited into primary module 210. Alternatively or additionally, circuitry 230 can receive information (such as the regimen, e.g.) remotely and/or from user 260.

Inventory manager 280 includes a medication or other prescribed component (C₁) 281, for example, and can include one or more nutraceutical-containing or other components (C₂) 282. Inventory manager 280 can be accessible to user 260 via optional interface 262, which can optionally be used for receiving user input in lieu of interface 238. Alternatively or additionally, interface 262 can comprise a vending-machine-style dispenser able to dispense one or more of the component(s) 281, 282 to user 260. Even in an embodiment in which dispensations are via a third party, and in lieu of interface 262, primary module 210 can register the dispensation such as via linkage 232 or interface 238, and respond accordingly (by operation 150, e.g.).

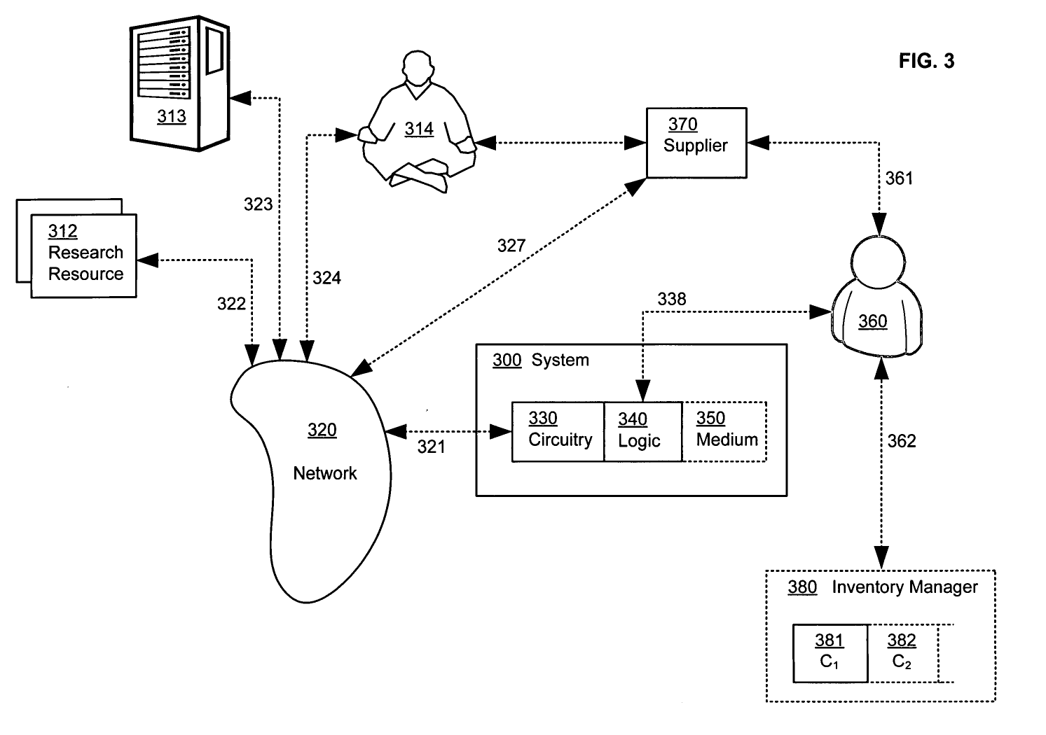

Referring now to FIG. 3, there is shown another example system 300 able to perform operational flow 100 of FIG. 1 and many similar variations. System 300 includes circuitry 330 and logic 340 as described below, and can further include one or more of link 321, interface 338, or medium 350. Logic 340 can optionally receive input from a user (such as from user 360 via interface 338, e.g.) locally. Medium 350 can bear one or more instructions that can be executed by logic 340 (optionally a computing device, e.g.) for performing one or more of the flows of FIGS. 5, 7, 8, 10, and 12.

As shown, link 321 can operably couple system 300 with network 320. In some embodiments, network 320 can thus have access to online research resource 312 through linkage 322 or to server 313 through linkage 323. Alternatively or additionally, network 320 can have access to expert 314 through linkage 324 or to supplier 370 through linkage 327. (“Expert” 314 can actually be an herbalist, a pharmacist, a physician, a psychologist, a parent, an author, a document, a database, a blog, or any other source of opinion or information.) Research resource 312 can be remote from system 300 or from server 313, expert 314, or supplier 370. Expert 314 can optionally be located at a clinic or similar retail or healthcare facility that can advantageously include system 300 as well as one or more of online research resource 312, server 313, supplier 370, or user 360.

System 300 optionally includes circuitry (optionally circuitry 330 with link 321, e.g.) for communicating with supplier 370 via network 320, such as by electronic mail, facsimile, or a similar digital format. Alternatively or additionally, circuitry 330 can coordinate with link 321 for communicating with a regimen server (such as server 313, e.g.) or with a consultant (such as expert 314) remotely. Alternatively or additionally, supplier 370 can be configured to communicate with expert 314 via linkage 327 or with (another) user 360 via linkage 361. Alternatively or additionally, user 360 can access a component C₁ 381 (and optionally one or more additional components C₂ 382) in an inventory manager 380 via interface 362.

In some embodiments, system 300 is a computer or similar device with inventory maintenance capabilities. In performing operation 140 (of FIG. 1) or some variants described below, logic 340 can register a dispensation from an inventory, for example by receiving an indication of the dispensation via user interface 338. Circuitry 330 can respond as appropriate by indicating a request quantity (at operation 150, e.g.), such as by transmitting the request quantity via link 321 as an order (to supplier 370, e.g.) or as a validation request (to a physician or other expert 314, e.g.).

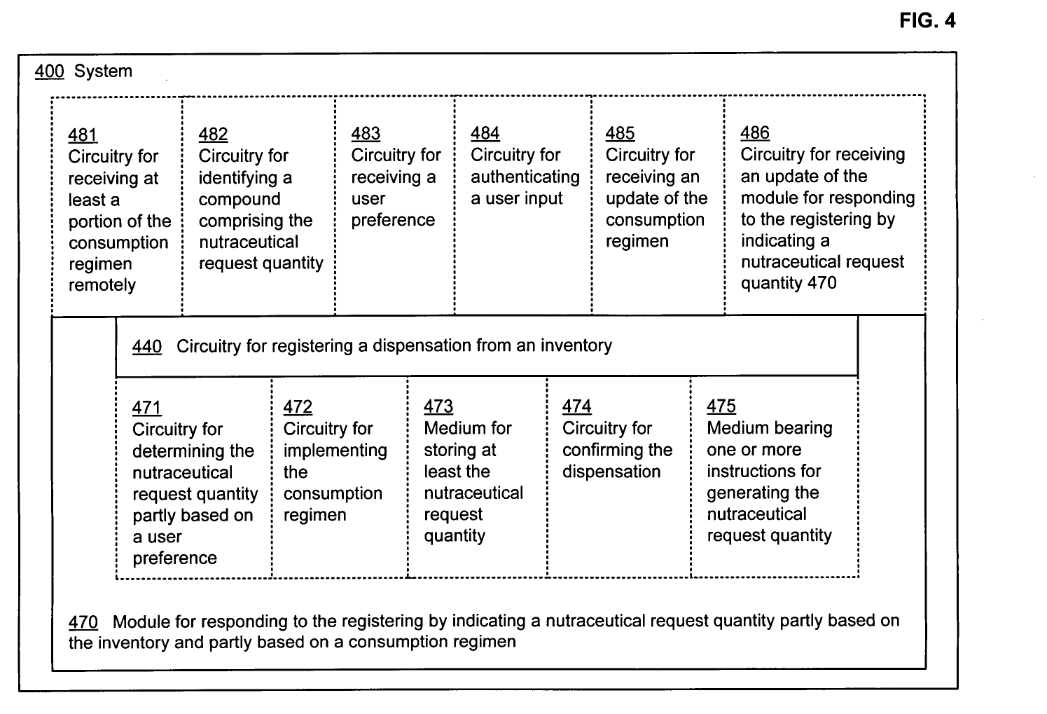

Referring now to FIG. 4, there is shown another example system 400 able to perform operational flow 100 of FIG. 1 and many similar variations such as those of FIGS. 5, 7, 8, 10, and 12. System 400 comprises circuitry 440 for registering a dispensation from an inventory and module 470 for responding to the registering by indicating a nutraceutical request quantity partly based on the inventory and partly based on a consumption regimen. Circuitry 440 can adjoin, overlap, or otherwise be operable to interact with module 470.

Module 470 optionally comprises circuitry 471 for determining the nutraceutical request quantity partly based on a user preference. Alternatively or additionally, module 470 can comprise one or more of circuitry 472 for implementing the consumption regimen, medium 473 for storing at least the nutraceutical request quantity, circuitry 474 for confirming the dispensation, or medium 475 bearing one or more instructions for generating the nutraceutical request quantity.

Also as shown, alternatively or additionally, system 400 can further comprise circuitry 481 for receiving at least a portion of the consumption regimen remotely. Alternatively or additionally, system 400 can further comprise circuitry 482 for identifying a compound comprising the nutraceutical request quantity, circuitry 483 for receiving a user preference, circuitry 484 for authenticating a user input, circuitry 485 for receiving an update of the consumption regimen, or circuitry 486 for receiving an update of the module for responding to the registering by indicating a nutraceutical request quantity 470.

Referring now to FIG. 5, there are shown various optional features of operational flow 100 of FIG. 1. In various implementations, system 400 of FIG. 4 can optionally be configured to perform flow 100 with one or more of operation 543, operation 551, operation 552, operation 556, operation 557, operation 558, or operation 560. Operation 543 comprises confirming the dispensation, such as can be performed by a module 470 that includes circuitry 474 of FIG. 4. Operation 551 comprises receiving a user preference, such as can be performed by a system 400 that includes circuitry 483 of FIG. 4. Operation 552 comprises identifying a compound comprising the nutraceutical request quantity, such as can be performed by a system 400 that includes circuitry 482 of FIG. 4. Operation 556 comprises determining the nutraceutical request quantity partly based on a user preference, such as can be performed by a module 470 that includes circuitry 471 of FIG. 4. Operation 558 comprises storing the nutraceutical request quantity, such as can be performed with medium 473 of FIG. 4 or with medium 350 of FIG. 3. These or other media of system 400 can likewise store data related to the request quantity, of course, such as other request quantities, medical history data, security information, supplier identification, user preferences, or instructions in light of teachings herein.

Operation 557 comprises receiving the nutraceutical request quantity, such as can be performed by certain embodiments of module 470. The request quantity can be received by the circuitry 481 for receiving at least a portion of the consumption regimen, for example, in which the portion includes a given quantity. The request quantity can likewise be received from module 470 after executing the one or more instructions for generating the request quantity borne by medium 475.

Operation 560 comprises performing one or more additional operations such as operation 561, operation 562, operation 563, operation 564, or operation 566. Operation 561 comprises receiving an update of the consumption regimen remotely, such as was described in the preceding paragraph. Operation 562 comprises scheduling the consumption regimen, such as can be performed by a module 470 that includes circuitry 472 of FIG. 4. Operation 563 comprises receiving an update of a module, such as can be performed by a system 400 that includes circuitry 486 of FIG. 4. Operation 564 comprises receiving at least a portion of the consumption regimen, such as can be performed by circuitry 481 of FIG. 4 or by logic 240 of FIG. 2, in some embodiments. Operation 566 comprises authenticating a user input, such as can be performed by a system 400 that includes circuitry 484 of FIG. 4.

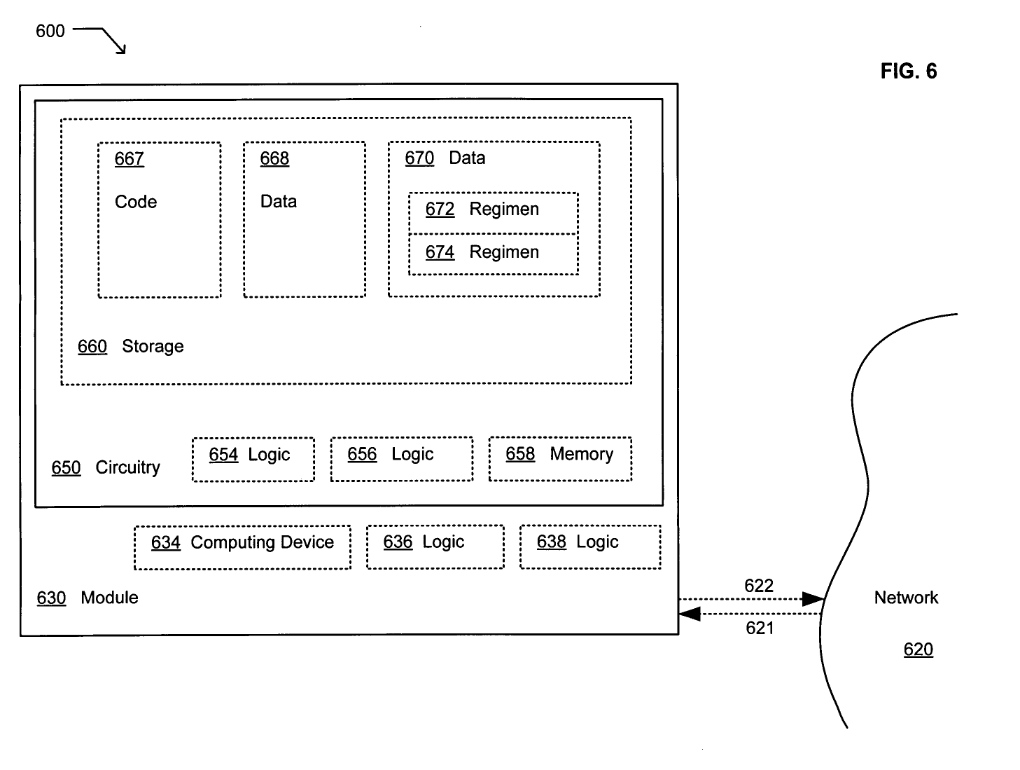

Referring now to FIG. 6, there is shown another example system 600 able to perform many variants of flow 100 described with reference to one or more of FIGS. 5, 7, 8, 10, and 12. System 600 can be configured to perform these variants with or without proximity or direct interaction with any user or inventory manager, except those few variants for which context dictates otherwise.

System 600 includes circuitry 650 for registering a dispensation from an inventory within a module 630 for responding to the registering by indicating a request, consistent with flow 100 of FIG. 1. As shown, module 630 can also include one or more of computing device 634, logic 636, or logic 638. Circuitry 650 can optionally include one or more of logic 654, logic 656, or memory 658. Storage 660 can optionally include one or more of code 667, data 668 (which can comprise historical data or inventory data, e.g.), or other data 670 such as one or more regimens 672, 674.

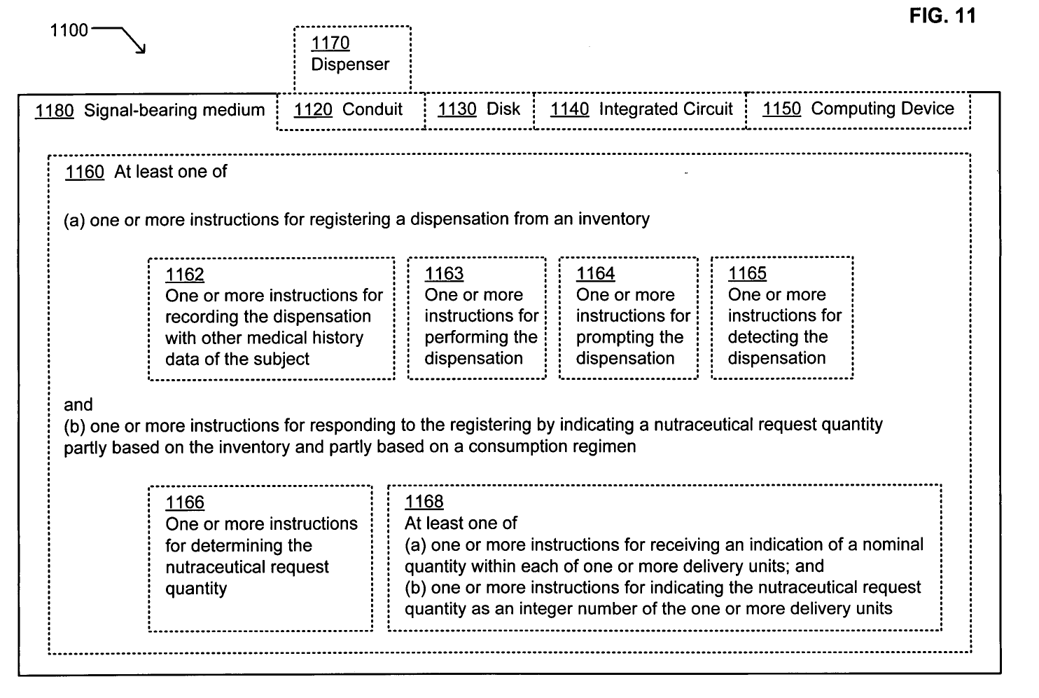

In some embodiments, logic 654 can comprise logic for prompting the test result (and/or other information that may relate to the subject) by requesting a test that partly depends on a medical history of the subject, for example, by transmitting a prompting signal as output 622 to network 620. (See FIGS. 11&12.) Alternatively or additionally, system 600 can perform a flow 100 including an operation 556, for example, based on a user preference received via input 621 and archived in storage 660.

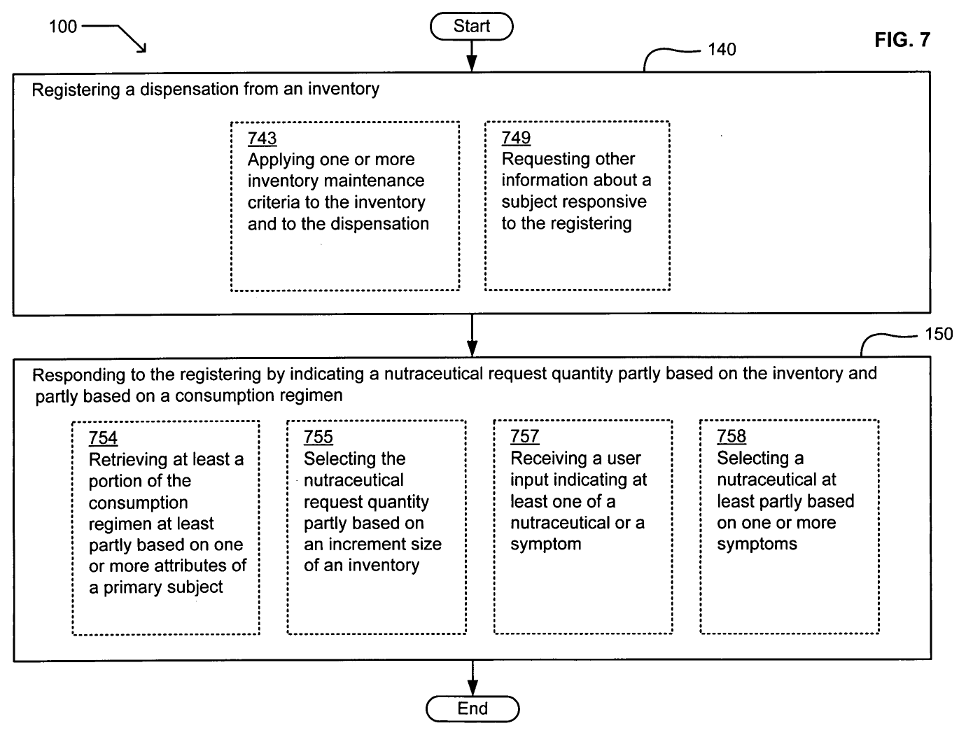

Referring now to FIG. 7, there are shown various optional features of operational flow 100 of FIG. 1 or 5. In various implementations, system 600 of FIG. 6 can optionally be configured to perform flow 100 with one or more of operation 743 or operation 749.

Operation 743 comprises applying one or more inventory maintenance criteria to the inventory and to the dispensation. The criteria can include a default or user-specified upper limit on a monetary value of an inventory, for example, or a maximum acceptable age of a perishable nutraceutical-containing compound. In response to receiving an input 621 indicating that a user's inventory manager is nearly full, for example, applying the criteria may accordingly reduce the request quantity to avoid spoilage, substantial investment in an ineffective or unneeded regimen, or other forms of waste. Also, system 600 optionally includes logic 638 comprising logic for requesting other information about a subject responsive to the registering (at operation 749), such as by transmitting one or more requests as output 622 to network 620. This approach can help tailor the regimen by seeking input when a user is likely to be available, for example, around the time of the registering.

Operation 754 comprises retrieving at least a portion of the consumption regimen at least partly based on one or more attributes of a primary subject. If a user responds to the requesting operation by identifying the subject of operation 749 as the primary subject, for example, operation 754 can include an operation of retrieving a regimen (regimen 672, e.g.) unique to the primary subject. Absent such an individualized regimen, even a few attributes such as age, gender, weight, and symptom can form a basis for an effective regimen.

Operation 755 comprises selecting the nutraceutical request quantity partly based on an increment size of an inventory. The request quantity may be expressed as an integer, for example, to identify a number of bottles or other containers selected by operation 755. Data 668 may identify the increment size, for example, used for performing the selecting operation 755.

Operation 757 comprises receiving a user input indicating at least one of a nutraceutical or a symptom. The user input may be received via a questionnaire (electronic or paper, e.g.), for example. Logic 656 can optionally be configured for selecting a nutraceutical at least partly based on one or more symptoms, for example, performing operation 758 responsive to detecting a user response indicating the one or more symptoms.

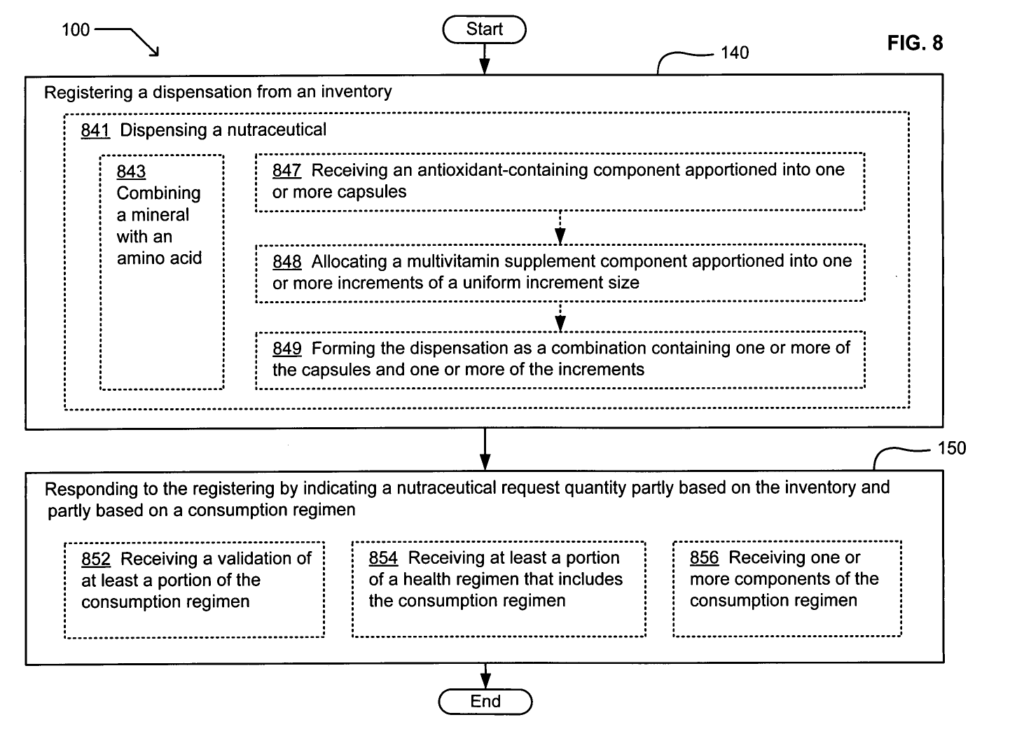

Referring now to FIG. 8, there are shown various optional features of operational flow 100 of FIG. 1, 5, or 7. In various implementations, system 200 of FIG. 2 can optionally be configured to perform flow 100 with operation 140 including one or more of operation 841, operation 843, operation 847, operation 848, or operation 849.

Operation 841 comprises dispensing a nutraceutical. Inventory manager 280 can perform this operation, for example, by dispensing a nutraceutical-containing component 282 responsive to an instruction (via linkage 232) from primary module 210. The registering itself can cause or enable or result from the dispensing operation 841, or stand in some other relation to the dispensing operation 841, conditional or otherwise.

Operation 843 comprises combining a mineral with an amino acid, optionally by encapsulating them in a pharmaceutically acceptable buffer. For a subject who takes a steady regimen including more than one component (such as these), such encapsulation is a convenient mode of performing operation 841.

Alternatively, a nutraceutical-containing dispensation can be prepared by combining more discrete increments. As shown, for example, operation 847 of receiving an antioxidant-containing component apportioned into one or more capsules can be combined with operation 848 of allocating a multivitamin supplement component apportioned into one or more increments of a uniform increment size and operation 849 of forming the dispensation as a combination containing one or more of the capsules and one or more of the increments. Combining discrete increments can be convenient for implementing a conditional regimen (or adjusting a stable regimen) by prompting actions in a user's proximity.

FIG. 8 also shows various optional features of operational flow 100 of FIG. 1, 5, or 7 comprising additional operation 852, operation 854, or operation 856. In various implementations, system 300 of FIG. 3 can optionally be configured to perform flow 100 with operation 150 including one or more of these additional operations.

Operation 852 comprises receiving a validation of at least a portion of the consumption regimen. The validation can be received from user 360 or expert 314, for example, optionally in response to a request for such a validation. The request can define or justify the portion of the consumption regimen briefly, for example, and specify an action (such as clicking a button or entering a code, e.g.) by which the validation can be generated. The responding operation 150 can optionally be made conditional on receiving the validation before a deadline, for example, or can otherwise affect a condition or manner by which system 300 will complete the responding operation 150.

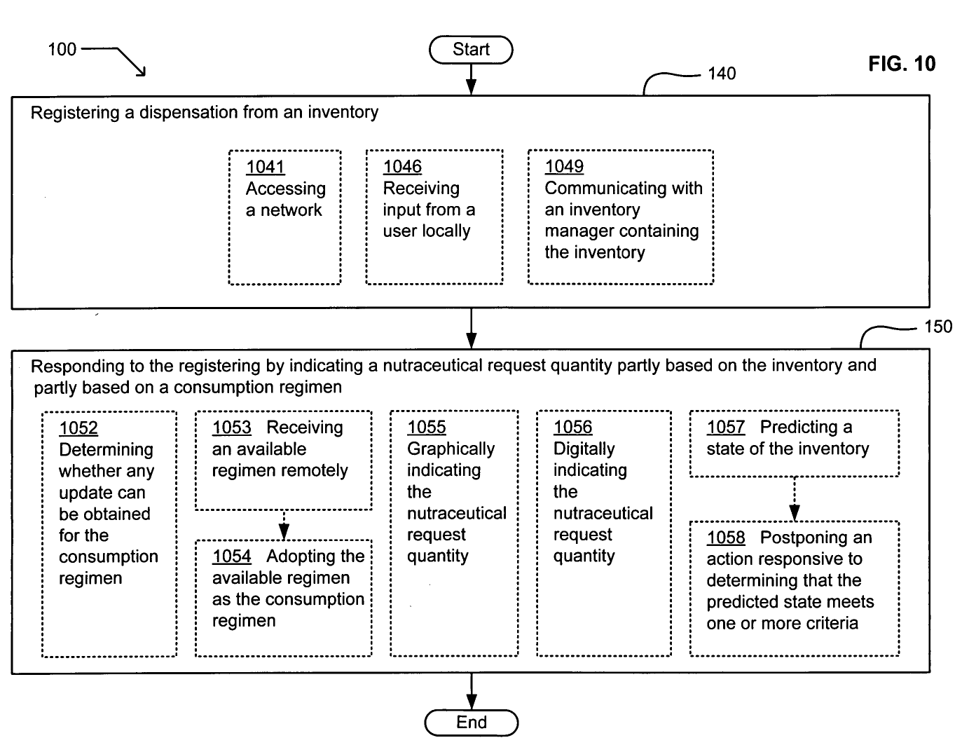

Operation 854 comprises receiving at least a portion of a health regimen that includes the consumption regimen. The portion can be received by circuitry 330 via network 320, for example. System 300 can present several such health regimens of interest to user 360 via interface 338, for example, prompting user 360 to adopt one or more of the health regimens. Logic 340 can then implement one or more user-adopted regimens by storing one or more instructions of each of the adopted regimens in medium 350 (a memory, e.g.). In this manner system 300 can perform operation 856 of receiving one or more components of the consumption regimen before, during, after, or without the above-described adoption operation. See FIG. 10, for example.

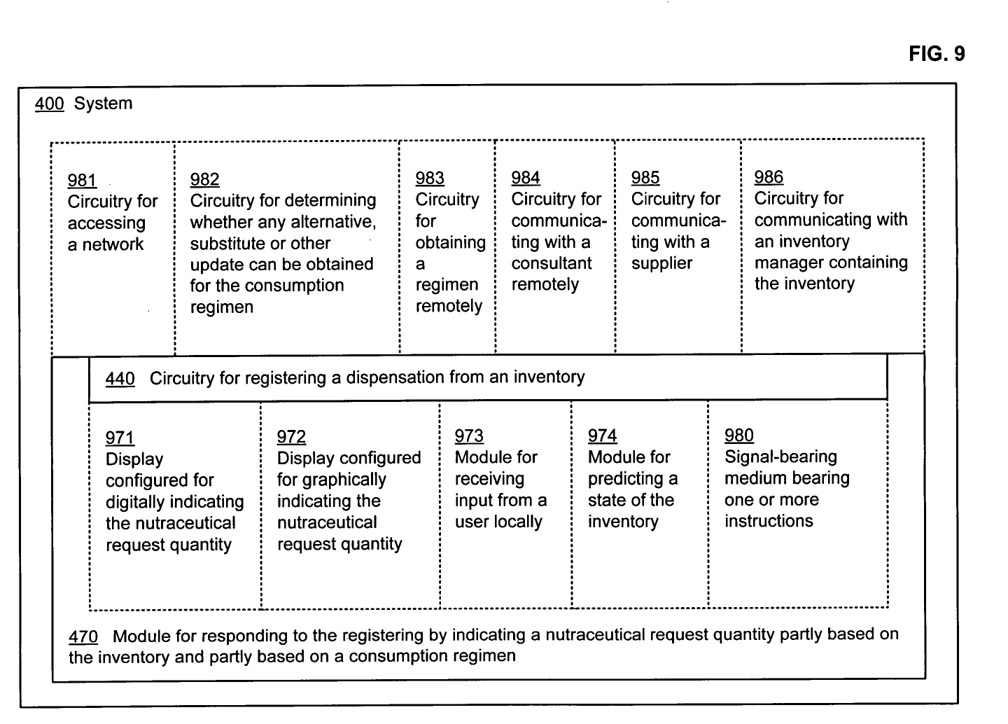

Referring now to FIG. 9, there are shown further optional features of system 400 of FIG. 4. As explained above, system 400 comprises circuitry 440 for registering a dispensation from an inventory and module 470 for responding to the registering by indicating a nutraceutical request quantity partly based on the inventory and partly based on a consumption regimen.

Module 470 optionally comprises display 971 configured for digitally indicating the nutraceutical request quantity. Alternatively or additionally, module 470 can comprise one or more of display 972 configured for graphically indicating the nutraceutical request quantity, module 973 for receiving input from a user locally, or module 974 for predicting a state of the inventory. Alternatively or additionally, module 470 can comprise signal-bearing medium 980 bearing one or more instructions such as those of FIG. 11 below.

Also as shown, alternatively or additionally, system 400 can further comprise circuitry 981 for accessing a network. Alternatively or additionally, system 400 can further comprise circuitry 982 for determining whether any update can be obtained for the consumption regimen, circuitry 983 for obtaining a regimen remotely, circuitry 984 for communicating with a consultant remotely, circuitry 985 for communicating with a supplier, or circuitry 986 for communicating with an inventory manager containing the inventory.

Referring now to FIG. 10, there are shown various optional features of operational flow 100 of FIG. 1, 5, 7 or 8. In various implementations, system 400 of FIG. 9 can optionally be configured to perform flow 100 with operation 140 including one or more of operation 1041, operation 1046, or operation 1049. Operation 1041 comprises accessing a network, such as by circuitry 981 in the system 400 as shown in FIGS. 4 & 9. Operation 1046 comprises receiving input from a user locally module 973. Operation 1049 comprises communicating with an inventory manager containing the inventory, such as by circuitry 986.

Operation 1052 comprises determining whether any alternative, substitute, or other update can be obtained for the consumption regimen, such as can be performed by certain embodiments of module 470 comprising circuitry 982 for determining whether any alternative, substitute, or other update can be obtained for the consumption regimen. The determining can be performed by checking a mailbox, for example, or otherwise by sending a message into a network such as may be received by a server. Making a negative determination can be performed by receiving a negative reply or by waiting a given duration without receiving a reply, for example, or by making some similarly reasonable inference derived from one or more received signals.

Operation 1053 comprises receiving an available regimen remotely, such as can be performed by certain embodiments of system 400 comprising circuitry 983. Circuitry 983 can receive the available regimen in response to a request or subscription, for example, or may select the available regimen as a preferable regimen using information about the subject. Operation 1054 comprises adopting the available regimen as the consumption regimen, such as can be performed by certain embodiments of system 400 comprising module 973, optionally responsive to a selection or other validation from the subject or some other user or consultant.

Operation 1055 comprises graphically indicating the nutraceutical request quantity, such as can be performed by certain embodiments of system 400 comprising display 972. Display 972 can optionally show a first component that is visually distinguishable from at least a second component, such as by color or shape.

Operation 1056 comprises digitally indicating the nutraceutical request quantity, such as can be performed by certain embodiments of system 400 comprising display 971. Alternatively or additionally, indicating operation 1056 can be performed by transmitting the nutraceutical request quantity digitally via a conduit (of FIG. 11, e.g.) or a wireless link (of FIG. 3, e.g.).

Operation 1057 comprises predicting a state of the inventory, such as can be performed by certain embodiments of system 400 comprising module 974. The prediction may be based on one or more attributes of one or more dispensations such as the registered one(s), for example, or upon the consumption regimen or a current state of the inventory. The predicted state may be detailed, such as a complete description of each component and its ingredients, its age, its location, and the like. The predicted state may alternatively be more basic, such as “adequate” or “in need of component Z.” The predicted state may also be expressed in various forms, such as a duration or a percentage. Part or all of the predicted state can be used for a variety of purposes such as operation 1058 of postponing an action responsive to determining that the predicted state meets one or more criteria. Involving a user can be postponed, for example, responsive to a predicted state of “X days of inventory adequacy,” for example.

Referring now to FIG. 11, a system 1100 includes a signal-bearing medium 1180 that can comprise (or interact with) a conduit 1120, a disk 1130, an integrated circuit 1140, or a computing device 1150. System 1100 can further include a dispenser 1170. Medium 1180 can optionally bear one or more instructions 1160 comprising at least one of (a) one or more instructions for registering a dispensation from an inventory and (b) one or more instructions for responding to the registering by indicating a nutraceutical request quantity partly based on the inventory and partly based on a consumption regimen.

Alternatively or additionally, medium 1180 can bear one or more instructions 1162 for recording the dispensation with other medical history data of the subject. The other medical history data can include one or more prior recordings of a dispensation, for example. Alternatively or additionally, medium 1180 can bear one or more instructions 1163 for performing the dispensation. Alternatively or additionally, medium 1180 can bear one or more instructions 1164 for prompting the dispensation. Alternatively or additionally, medium 1180 can bear one or more instructions 1165 for detecting the dispensation. Alternatively or additionally, medium 1180 can bear one or more instructions 1166 for determining the nutraceutical request quantity. Alternatively or additionally, medium 1180 can bear one or more instructions 1168 comprising at least one of (a) one or more instructions for receiving an indication of a quantity within each of one or more delivery units and (b) one or more instructions for indicating the nutraceutical request quantity as an integer number of the one or more delivery units. A given set of one or more instructions can comprise more than one of instructions 1162-1168, of course, optionally executable by computing device 1150.

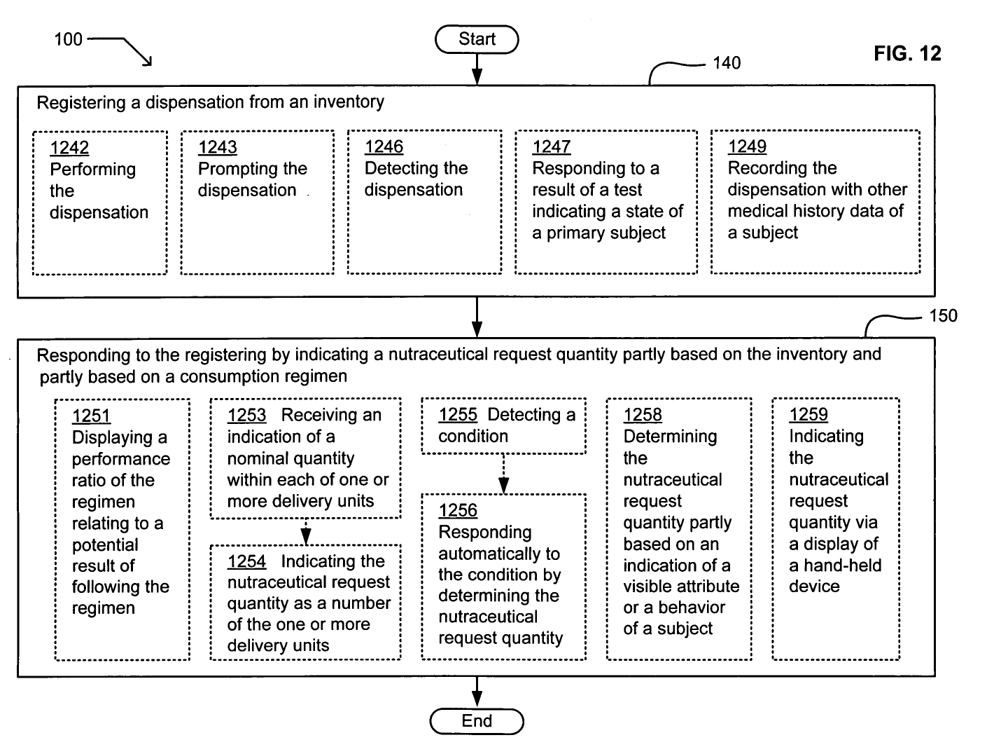

Referring now to FIG. 12, there are shown various optional features of operational flow 100 of FIG. 1, 5, 7, 8, or 10. In various implementations, system 1100 of FIG. 11 can optionally be configured to perform flow 100 with operation 140 including one or more of operation 1242, operation 1243, operation 1246, operation 1247, or 1249.

Operation 1242 comprises performing the dispensation, such as by an embodiment of system 1100 in which one or more instructions 1163 are transmitted via medium 1180 to dispenser 1170.

Operation 1243 comprises prompting the dispensation. This can be performed by a computing device 1150, for example, such as by transmitting a dispensing command or by otherwise causing an actuation of a dispenser. Operation 1246 comprises detecting the dispensation. This can be performed by integrated circuit 1140, for example, before, during, after, or without the dispensation. The dispensation to be registered can optionally be conditional, scheduled, or actual.

Operation 1247 comprises responding to a result of a test indicating a state of a subject. This can be performed by executing one or more instructions 1166 for determining the nutraceutical request quantity, for example, in some embodiments of system 1100. Operation 1249 comprises recording the dispensation with other medical history data of a subject, such as by one or more instructions 1162.

Operation 1251 comprises displaying a performance ratio of the regimen relating to a potential result of following the regimen. The potential result can be tangible, intangible, subjective, objective, or some combination of these. The performance characteristic(s) can comprise an estimated probability of a measurable improvement, a ratio of “satisfied” regimen subscribers to “dissatisfied” regimen subscribers, a record of subjective experience, an anecdotal log, a rating, a research summary, a certification, an endorsement, or some other indicator.

Operation 1253 comprises receiving an indication of a quantity within each of one or more delivery units. The quantity can be a nominal increment of mass (such as milligrams of a vitamin, e.g.), a count (of eggs, e.g.), a length, or some other convenient increment. In some embodiments, a code module comprising one or more instructions 1166 performs operation 1253 before or during operation 1254 of indicating the nutraceutical request quantity as a number of the one or more delivery units.

Operation 1255 comprises detecting a condition. The condition can comprise a substantially periodic event, for example, such as an appointment, a day of the month, a time of day, a mealtime, a work break, or the like. The condition can also comprise a storm or other weather condition, a job change or price change or other economic event, a smoking habit or some other personal condition, or any other condition that may affect the subject's life.

The detecting can be performed by a calendar program, an alarm clock, a thermometer, or a real time trigger, for example. In some variants of embodiments described above, system 300 of FIG. 3 performs operation 150 responsive to one or more criteria that depend on the detected event(s) as well as the request quantity and the regimen. A care provider can use system 300 to adjust or otherwise update a regimen-implementing request automatically, responsive to a scheduled visit by a patient, for example, if system 300 performs operation 1256 of responding to the condition by determining the nutraceutical request quantity.

Alternatively or additionally, a subject or care provider may instruct system 300 to refine the subject's profile responsive to an event, such as by operation 1258 of determining the nutraceutical request quantity partly based on an indication of a visible attribute or a behavior of a subject. The subject, a psychologist, a psychiatrist, a parent, or some other observer may provide an objective indication (conscious, e.g.), for example, or a subjective indication (irritable or fatigued, e.g.) of the behavior. The request quantity may be zero, for example, if the visible attribute or the behavior indicates a negative reaction by the subject to a component of that regimen, for example. The indication may relate to a facial attribute (“bags under the eyes,” e.g.), some other skin condition (“rash,” e.g.), or any other visible attribute of a subject. A message may be received via link 321, for example, including the indication and an identification of the observer. The message may, of course, include one or more other test results also.

Operation 1259 comprises indicating the nutraceutical request quantity via a display of a hand-held device. Any of the above-described systems can optionally include a display, for example. Alternatively or additionally, the system can comprise a hand-held device such as a cell phone, wrist watch or the like.

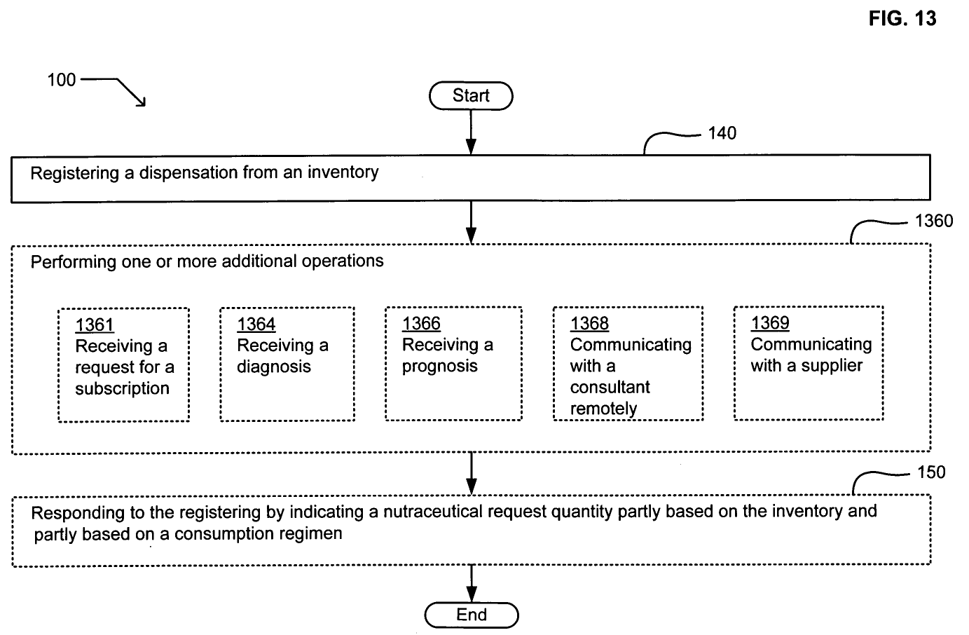

Referring now to FIG. 13, there are shown various optional features of operational flow 100 of FIG. 1, 5, 7, 8, 10, or 12. In various implementations, system 600 of FIG. 6 can optionally be configured to perform flow 100 by performing one or more additional operations 1360 of operation 1361, operation 1364, operation 1366, operation 1368, or 1369.

Operation 1361 comprises receiving a request for a subscription, such as may be performed by logic 656 configured as logic for receiving input 621. In an embodiment in which code 667 includes inventory maintenance software, for example, logic 656 can respond to the request by selling a subscription or similar license. Logic 656 can likewise respond partly based on one or more factors such as the code 667, a regimen 674 selected by a user, a location of the user's system, data 668 such as a description of inventories within a given range of the user, one or more user preferences such as cost, or one or more objective indications such as symptoms indicated by the user.

Operation 1364 comprises receiving a diagnosis. Operation 1366 comprises receiving a prognosis. The diagnosis or prognosis can optionally be based on one or more test results and/or reports by others, observations, predictions, reports, history, or other indications of status). The diagnosis or prognosis may relate to a subject who has adopted the consumption regimen, for example, and may warrant storing the diagnosis in a medical history portion of data 670. The subject may be diagnosed as having an allergy to seafood, for example, which may bear against a fish oil supplement. Substantially the same criterion (“no fish,” e.g.) may effectively become a part of a given subject's regimen (regimen 672, e.g.) in other circumstances as well. It may arise from a received user instruction or preference (“no fish,” e.g.), a user status (“vegan,” e.g.), or otherwise arise from a received input 621. The subject may be diagnosed with osteoporosis, as another example, which may bear toward a calcium-containing (or higher-calcium) regimen. Many embodiments described herein allow for a more sophisticated and cost-effective approach to building and maintaining an inventory of effective remedies.

Logic 638 can optionally perform one or more of operation 1368 of communicating with a consultant remotely and/or operation 1369 of communicating with a supplier. These operations can generate timely information about the availability or advisability of an available regimen, for example, facilitating appropriate adjustments at operation 150.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Moreover, “can” and “optionally” and other permissive terms are used herein for describing optional features of various embodiments. These terms likewise describe selectable or configurable features generally, unless the context dictates otherwise.

The herein described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interactable and/or logically interacting components.

While certain features of the described implementations have been illustrated as disclosed herein, many modifications, substitutions, changes and equivalents will now occur to those skilled in the art. It is, therefore, to be understood that the appended claims are intended to cover all such modifications and changes as fall within the true spirit of the embodiments of the invention.

1. A method comprising:
registering a dispensation from an inventory; and
responding to the registering by indicating a nutraceutical request quantity partly based on the inventory and partly based on a consumption regimen.

2. The method of claim 1, wherein registering a dispensation from an inventory comprises:
confirming the dispensation.

3. (canceled)

4. The method of claim 1, wherein responding to the registering by indicating a nutraceutical request quantity partly based on the inventory and partly based on a consumption regimen comprises:
identifying a compound comprising the nutraceutical request quantity.

5. (canceled)

6. The method of claim 1, wherein responding to the registering by indicating a nutraceutical request quantity partly based on the inventory and partly based on a consumption regimen comprises:
determining the nutraceutical request quantity partly based on a user preference.

7. (canceled)

8. The method of claim 1, wherein responding to the registering by indicating a nutraceutical request quantity partly based on the inventory and partly based on a consumption regimen comprises:
storing the nutraceutical request quantity.

9. (canceled)

10. (canceled)

11. (canceled)

12. The method of claim 1, further comprising: scheduling the consumption regimen.

13. The method of claim 1, further comprising:
receiving an update of the consumption regimen remotely; and
scheduling the consumption regimen.

14. The method of claim 1, further comprising:
receiving an update of a module.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. The method of claim 1, wherein responding to the registering by indicating a nutraceutical request quantity partly based on the inventory and partly based on a consumption regimen comprises:
receiving an available regimen remotely; and
adopting the available regimen as the consumption regimen.

42. The method of claim 1, wherein responding to the registering by indicating a nutraceutical request quantity partly based on the inventory and partly based on a consumption regimen comprises:
graphically indicating the nutraceutical request quantity.

43. (canceled)

44. The method of claim 1, wherein responding to the registering by indicating a nutraceutical request quantity partly based on the inventory and partly based on a consumption regimen comprises:
predicting a state of the inventory.

45. (canceled)

46. (canceled)

47. The method of claim 1, wherein registering a dispensation from an inventory comprises:
prompting the dispensation.

48. (canceled)

49. The method of claim 1, wherein registering a dispensation from an inventory comprises:
responding to a result of a test indicating a state of a subject.

50. The method of claim 1, wherein registering a dispensation from an inventory comprises:
recording the dispensation with other medical history data of a subject.

51. (canceled)

52. The method of claim 1, wherein responding to the registering by indicating a nutraceutical request quantity partly based on the inventory and partly based on a consumption regimen comprises:
receiving an indication of a quantity within each of one or more delivery units.

53. (canceled)

54. (canceled)

55. (canceled)

56. (canceled)

57. (canceled)

58. A system comprising:
means for registering a dispensation from an inventory; and
means for responding to the registering by indicating a nutraceutical request quantity partly based on the inventory and partly based on a consumption regimen.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. The system of claim 58, wherein the means for registering a dispensation from an inventory comprises:
means for requesting other information about a subject responsive to the registering.

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. The system of claim 58, wherein the means for registering a dispensation from an inventory comprises:
means for prompting the dispensation.

101. (canceled)

102. (canceled)

103. (canceled)

104. The system of claim 58, wherein the means for responding to the registering by indicating a nutraceutical request quantity partly based on the inventory and partly based on a consumption regimen comprises:
means for displaying a performance ratio of the regimen relating to a potential result of following the regimen.

105. The system of claim 58, wherein the means for responding to the registering by indicating a nutraceutical request quantity partly based on the inventory and partly based on a consumption regimen comprises:
means for receiving an indication of a quantity within each of one or more delivery units.

106. (canceled)

107. (canceled)

108. The system of claim 58, wherein the means for responding to the registering by indicating a nutraceutical request quantity partly based on the inventory and partly based on a consumption regimen comprises:
means for determining the nutraceutical request quantity partly based on an indication of a visible attribute or a behavior of a subject.

109. The system of claim 58, wherein the means for responding to the registering by indicating a nutraceutical request quantity partly based on the inventory and partly based on a consumption regimen comprises:
means for indicating the nutraceutical request quantity via a display of a hand-held device.

110. A system comprising:
circuitry for registering a dispensation from an inventory; and
module for responding to the registering by indicating a nutraceutical request quantity partly based on the inventory and partly based on a consumption regimen.

111. (canceled)

112. (canceled)

113. (canceled)

114. The system of claim 110, wherein the module for responding to the registering by indicating a nutraceutical request quantity partly based on the inventory and partly based on a consumption regimen comprises:
circuitry for confirming the dispensation.

115. (canceled)

116. The system of claim 110, further comprising:
circuitry for receiving at least a portion of the consumption regimen remotely.

117. The system of claim 110, further comprising:
circuitry for identifying a compound comprising the nutraceutical request quantity.

118. The system of claim 110, further comprising:
circuitry for receiving a user preference.

119. The system of claim 110, further comprising:
circuitry for authenticating a user input.

120. The system of claim 110, further comprising:
circuitry for receiving an update of the consumption regimen.

121. (canceled)

122. The system of claim 110, wherein the module for responding to the registering by indicating a nutraceutical request quantity partly based on the inventory and partly based on a consumption regimen comprises:
a display configured for digitally indicating the nutraceutical request quantity.

123. The system of claim 110, wherein the module for responding to the registering by indicating a nutraceutical request quantity partly based on the inventory and partly based on a consumption regimen comprises:
a display configured for graphically indicating the nutraceutical request quantity.

124. The system of claim 110, wherein the module for responding to the registering by indicating a nutraceutical request quantity partly based on the inventory and partly based on a consumption regimen comprises:
a module for receiving input from a user locally.

125. The system of claim 110, wherein the module for responding to the registering by indicating a nutraceutical request quantity partly based on the inventory and partly based on a consumption regimen comprises:
a module for predicting a state of the inventory.

126. (canceled)

127. (canceled)

128. The system of claim 110, further comprising:
circuitry for determining whether any update can be obtained for the consumption regimen.

129. (canceled)

130. The system of claim 110, further comprising:
circuitry for communicating with a consultant remotely.

131. The system of claim 110, further comprising:
circuitry for communicating with a supplier.

132. The system of claim 110, further comprising:
circuitry for communicating with an inventory manager containing the inventory.

133. A system comprising:
a computing device; and
at least one of one or more instructions for registering a dispensation from an inventory;
and one or more instructions for responding to the registering by indicating a nutraceutical request quantity partly based on the inventory and partly based on a consumption regimen.

134. (canceled)

135. (canceled)

136. (canceled)

137. (canceled)

138. The system of claim 133, further comprising:
a dispenser.

139. (canceled)

140. (canceled)

141. (canceled)

142. The system of claim 133, further comprising:
one or more instructions for detecting the dispensation.

143. (canceled)

144. (canceled)

145. A computer program product comprising:
a signal-bearing medium bearing at least one of
one or more instructions for registering a dispensation from an inventory; and
one or more instructions for responding to the registering by indicating a nutraceutical request quantity partly based on the inventory and partly based on a consumption regimen.

146. (canceled)

147. The computer program product of claim 145, in which the signal-bearing medium comprises:
a disk.

148. (canceled)

149. (canceled)

150. (canceled)

151. (canceled)

152. (canceled)

153. (canceled)

154. (canceled)
